(19) United States
(12) Patent Application Publication
(10) Pub. No.: US 2022/0067928 A1
(43) Pub. Date: Mar. 3, 2022

(54) METHOD FOR LOCATING AND CHARACTERIZING BIFURCATIONS OF A CEREBRAL VASCULAR TREE, ASSOCIATED METHODS AND DEVICES

(71) Applicants: INSTITUT NATIONAL DE LA SANTE ET DE LA RECHERCHE MEDICALE (INSERM), Paris (FR); UNIVERSITE DE NANTES, Nantes (FR); CENTRE NATIONAL DE LA RECHERCHE SCIENTIFIQUE, Paris (FR)

(72) Inventors: Florent AUTRUSSEAU, Nantes (FR); Romain BOURCIER, Nantes (FR); Anass NOURI, Brest (FR)

(21) Appl. No.: 17/299,481

(22) PCT Filed: Dec. 4, 2019
(86) PCT No.: PCT/EP2019/083731

§ 371 Date: Jun. 3, 2021

(30) Foreign Application Priority Data
Dec. 4, 2018 (EP) 18306612.5

(51) Int. Cl. G06T 7/00; G16H 50/20; G16H 50/30

(52) U.S. Cl. CPC G06T 7/0012; G16H 50/20; G06T 2207/10028; G06T 2207/20072; G06T 2207/30101; G16H 50/30

(57) ABSTRACT

Technics are known to use vessel bifurcations detection to obtain access to the detection of aneurysms. However, such technics suffer from a relatively poor accuracy. Therefore, the Applicants have developed a specific method for locating and characterizing bifurcations of a cerebral vascular tree based on a graph analysis of a three-dimensional skeleton of the tree. This enables to determine more accurately the vessel bifurcations. Such property can be used advantageously for several applications such as predicting the risk of developing an aneurysm, diagnosing an aneurysm, identifying a therapeutic target, identifying a biomarker or screening a compound.

### TECHNICAL FIELD OF THE INVENTION

The present invention concerns a method for determining at least one bifurcation of a vascular tree. The invention concerns a method for predicting that a subject is at risk of developing an aneurysm. The invention also relates to a method for diagnosing an aneurysm. The invention also concerns a method for identifying a therapeutic target for preventing and/or treating an aneurysm. The invention also relates to a method for identifying a biomarker, the biomarker being a diagnostic biomarker of an aneurysm, a susceptibility biomarker of an aneurysm, a prognostic biomarker of an aneurysm or a predictive biomarker in response to the treatment of an aneurysm. The invention also concerns a method for screening a compound useful as a medicine, the compound having an effect on a known therapeutical target, for preventing and/or treating an aneurysm. The invention also relates to the associated computer program products and a computer readable medium.

### BACKGROUND OF THE INVENTION

The cardiovascular system (also called circulatory system) is composed of all blood vessels (arteries, capillaries and veins) that carry blood and lymph through the entire human body. The purpose of this organ system is to transport nutrients, oxygen and carbon dioxide between body tissues. On certain organs, such as the heart, the liver, the kidneys, the lungs or the brain, the vascular system becomes denser. When reaching these organs, the arteries, capillaries or veins split into several branches, this forms a vascular tree.

The circulatory system may undergo various vascular diseases, such as atherosclerosis, blood clots, inflammation or some genetic diseases. Several factors, such as smoking habits, hypertension, cardio vascular history or some particular treatments may lead to a weakened vascular system. The vascular diseases may occur on various arteries of the human body and thus induce different effects (such as coronary artery disease, thoracic vascular disease and abdominal aortic aneurysms).

A weakened wall of the blood vessel may lead to the formation of an aneurysm. In the brain, aneurysms may take several forms, frequently as dissecting aneurysms (blood leaking out of the inner layer of the artery wall), fusiform aneurysms (local bulging of the artery characterized by a ballooning of the vessel, i.e. a local increase of the diameter), or saccular (sometimes called berry) aneurysms (a bulge occurring on a single side of the artery). Ninety percent of the cerebral aneurysms belong to this latter form.

Often an aneurysm may remain benign and never evolve into a dangerous state. The main complication induced by an aneurysm is when it does rupture, then blood will then escape into the surrounding tissues and provoke a sub-arachnoid hemorrhage that may lead to the death or a permanent disability. The rupture causes a decreased blood flow downstream, and thus, an ischemia. ICAs must be closely monitored, as the risk of rupture is prevalent: the risk of rupture is higher along a sub set of arteries located in the centre of the brain called the “Circle of Willis”. Eighty-five percent of the saccular ICAs occur along the Circle of Willis. ICA aneurysms are quite prevalent, affecting 2 to 5 percent of the adult worldwide population. An ICA rupture happens to 40 about 8-10/100.000 persons per year for the Caucasian population and to about 20/100.000 persons per year for Japanese or Finnish populations.

It is therefore desirable to detect such pathologies and notably cerebrovascular diseases, and more specifically on the formation of Intra Cranial Aneurysms (ICA).

Several methods are known to use vessel bifurcations to obtain access to the detection of such pathologies.

In particular, an article by Macedo et al. entitled “A centerline-base estimator of vessel bifurcations in angiography images” and published in Medical Imaging 2013: Computer-Aided Diagnosis, Proc. of SPIE indicates that the analysis of vascular structure based on vessel diameters, density and distance between bifurcations is an important step towards the diagnosis of vascular anomalies. Moreover, vascular network extraction allows the study of angiogenesis. This article describes a technique that detects bifurcations in vascular networks in magnetic resonance angiography and computed tomography angiography images. Initially, a vessel tracking technique that uses the Hough transform and a matrix composed of second order partial derivatives of image intensity is used to estimate the scale and vessel direction, respectively. This semi-automatic technique is capable of connecting isolated tracked vessel segments and extracting a full tree from a vascular network with minimal user intervention. Vessel shape descriptors such as curvature are then used to identify bifurcations during tracking and to estimate the next branch direction. In this article, the authors have initially applied this technique on synthetic datasets and then on real images.

Another technique is also known from an article by Zhao and Hamarneh named “Bifurcation Detection in 3D Vascular Images Using Novel Feature and Random Forest” and extracted from IEEE International Symposium on Biomedical Imaging, p. 421 to 424 (2014). The authors explain that bifurcation detection is important in medical image analysis for mainly two reasons: 1) plaques are easy to accumulate at artery bifurcations, which leads to atherosclerosis and strokes; 2) for quantification (e.g. branch length, thickness, tortuosity), visualization, and blood flow simulation, it is necessary to extract all the branches and their connectivity in a vessel tree, which makes bifurcation localization crucial. In this article, several novel features are designed for classifying bifurcations in 3D vascular images using random forest. Encouraging results with both synthetic and real datasets are obtained.

However, in each of these previous techniques, the obtained precision is not satisfying.

### SUMMARY OF THE INVENTION

The invention aims at providing a method for determining at least one parameter of a bifurcation of a vascular tree that provides with a better precision.

To this end, the specification describes a method for determining at least one parameter of at least one bifurcation of a vascular tree of a subject, notably a cerebral one, the method being computer-implemented. The method comprises the steps of processing a three-dimensional image of the vascular tree with a first technique to obtain a three-dimensional skeleton of the tree, analyzing the obtained skeleton by a second technique to obtain a graph of the vascular tree, the graph being a set of nodes linked by edges with a weight, and detecting the presence of a bifurcation when the graph comprises a node linked to at least three edges.

According to further aspects of the invention which are advantageous but not compulsory, the method for determining might incorporate one or several of the following features, taken in any technically admissible combination:
the method for determining further comprises a step of determining a characteristic of the bifurcation.
a bifurcation angle is defined for a bifurcation, the step of determining comprising determining the bifurcation angle of the detected bifurcation.
the method further comprises a step of calculating the geodesic distance between two bifurcations.
a cross-section area is defined for a bifurcation, the step of determining comprising determining the cross-section area of the detected bifurcation.
the method comprises a step of obtaining the artery tortuosity, the step of obtaining comprising calculating the curvature of each voxel of an artery.
the step of obtaining comprises pooling the curvatures to obtain the tortuosity parameter.
the pooling is achieved by using a weighted Minkowski sum.

The specification describes a method for predicting that a subject is at risk of developing an aneurysm, the method for predicting at least comprising the step of carrying out the steps of a method for determining at least one parameter of at least one bifurcation of the vascular tree of the subject, to obtain determined parameters, the method for determining being as previously described and a step of predicting that the subject is at risk of developing the aneurysm based on the determined parameters.

The specification also relates to a method for diagnosing an aneurysm, the method for diagnosing at least comprising the step of carrying out the steps of a method for determining at least one parameter of at least one bifurcation of the vascular tree of the subject, the method for determining being as previously described, and diagnosing the aneurysm based on the determined parameters.

The specification also concerns a method for identifying a therapeutic target for preventing and/or treating an aneurysm, the method comprising at least the step of carrying out the steps of a method for determining at least one parameter of at least one bifurcation of the vascular tree of a first subject, to obtain first determined parameters, the method for determining being as previously described and the first subject being a subject suffering from the aneurysm, carrying out the steps of the method for determining at least one parameter of at least one bifurcation of the vascular tree of a second subject, to obtain second determined parameters, the method for determining being as previously described and the second subject being a subject not suffering from the aneurysm, and selecting a therapeutic target based on the comparison of the first and second determined parameters.

The specification describes a method for identifying a biomarker, the biomarker being a diagnostic biomarker of an aneurysm, a susceptibility biomarker of an aneurysm, a prognostic biomarker of an aneurysm or a predictive biomarker in response to the treatment of an aneurysm, the method comprising at least the step of carrying out the steps of a method for determining at least one parameter of at least one bifurcation of the vascular tree of a first subject, to obtain first determined parameters, the method for determining being as previously described and the first subject being a subject suffering from the aneurysm, carrying out the steps of the method for determining at least one parameter of at least one bifurcation of the vascular tree of a second subject, to obtain second determined parameters, the method for determining being as previously described and the second subject being a subject not suffering from the aneurysm, and selecting a biomarker based on the comparison of the first and second determined parameters.

The specification also relates to a method for screening a compound useful as a probiotic, a prebiotic or a medicine, the compound having an effect on a known therapeutical target, for preventing and/or treating an aneurysm, the method comprising at least the step of carrying out the steps of a method for determining at least one parameter of at least one bifurcation of the vascular tree of a first subject, to obtain first determined parameters, the method for determining being as previously described and the first subject being a subject suffering from the aneurysm and having received the compound, carrying out the steps of the method for determining at least one parameter of at least one bifurcation of the vascular tree of a second subject, to obtain second determined parameters, the method for determining being as previously described and the second subject being a subject suffering from the aneurysm and not having received the compound, and selecting a compound based on the comparison of the first and second determined parameters.

The specification also describes a computer program product comprising instructions for carrying out the steps of a method as previously described when said computer program product is executed on a suitable computer device.

The specification also relates to a computer readable medium having encoded thereon a computer program as previously described.

### BRIEF DESCRIPTION OF THE DRAWINGS

The invention will be better understood on the basis of the following description which is given in correspondence with the annexed figures and as an illustrative example, without restricting the object of the invention. In the annexed figures:

FIG. 1 shows schematically a system and a computer program product whose interaction enables to carry out a method for determining at least one bifurcation of a vascular tree,

FIG. 2 is a flowchart illustrating an example of carrying out of an example of a method for determining at least one bifurcation of a vascular tree,

FIG. 3 is a schematic view of an example of bifurcation,

FIGS. 4 and 5 are comparative graphs of the performance obtained in detecting bifurcations by several methods,

FIG. 6 is a graph showing the difference between two values for a first angle of bifurcation, one value being obtained by the method of FIG. 2 and the other value being measured manually by experts,

FIG. 7 is a graph showing the difference between two values for a second angle of bifurcation, one value being obtained by the method of FIG. 2 and the other value being measured manually by experts,

FIG. 8 is a graph showing the distribution of minimum and maximum diameters obtained by the method of FIG. 2 (left, two box plots) and obtained manually by experts (right, two boxplots), and

FIGS. 9 to 14 are graphs showing the distribution of various parameters of a bifurcation in the absence and in the presence of aneurysm.

### DETAILED DESCRIPTION OF SOME EMBODIMENTS

Description of the System

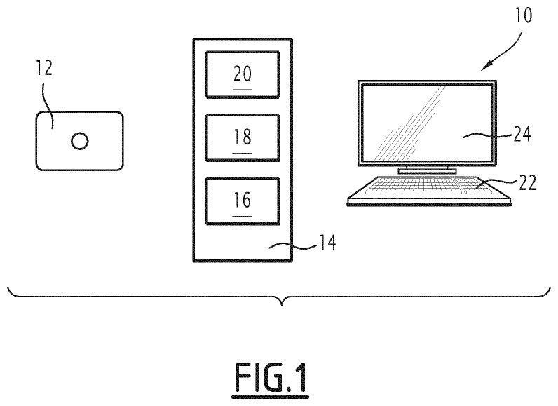

A system 10 and a computer program product 12 are represented in FIG. 1. The interaction between the computer program product 12 and the system 10 enables to carry out a method for determining at least one bifurcation of a cerebral vascular tree.

System 10 is a computer. In the present case, system 10 is a laptop.

More generally, system 10 is a computer or computing system, or similar electronic computing device adapted to manipulate and/or transform data represented as physical, such as electronic, quantities within the computing system's registers and/or memories into other data similarly represented as physical quantities within the computing system's memories, registers or other such information storage, transmission or display devices.

System 10 comprises a processor 14, a keyboard 22 and a display unit 24.

The processor 14 comprises a data-processing unit 16, memories 18 and a reader 20. The reader 20 is adapted to read a computer readable medium.

The computer program product 12 comprises a computer readable medium.

The computer readable medium is a medium that can be read by the reader of the processor. The computer readable medium is a medium suitable for storing electronic instructions, and capable of being coupled to a computer system bus.

Such computer readable storage medium is, for instance, a disk, a floppy disks, optical disks, CD-ROMs, magnetic-optical disks, read-only memories (ROMs), random access memories (RAMs) electrically programmable read-only memories (EPROMs), electrically erasable and programmable read only memories (EEPROMs), magnetic or optical cards, or any other type of media suitable for storing electronic instructions, and capable of being coupled to a computer system bus.

A computer program is stored in the computer readable storage medium. The computer program comprises one or more stored sequence of program instructions.

The computer program is loadable into the data-processing unit and adapted to cause execution of the method for determining when the computer program is run by the data-processing unit.

Operating of the System

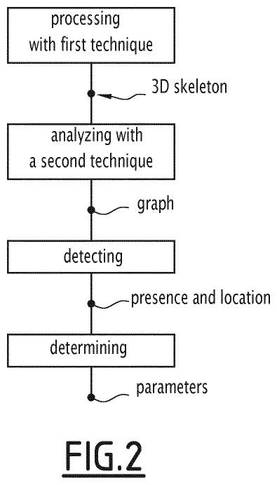

Operating of the system 10 is now described by illustrating an example of carrying out a method for determining at least one parameter of at least one bifurcation of the cerebral vascular tree as illustrated by the flowchart of FIG. 2.

A vascular tree is a set of blood arteries in an area. The cerebral vascular tree is the vascular tree of the brain of a subject.

It is to be noted that the cerebral vascular tree is only given as a specific example, bearing in mind that the method can be applied to any vascular tree.

It should also be mentioned that, contrary to many other methods belonging to the prior art, the method for determining which is detailed is able to handle three-dimensional vascular tree.

The subject is an animal, notably a mammal.

In particular, the subject is a mouse or a human being.

A bifurcation is a splitting of a mother artery into two or more daughter arteries.

In particular, it is not rare to find a trifurcation that is a splitting of a mother artery into three daughter arteries.

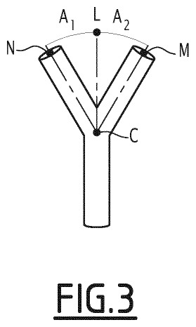

The meaning of bifurcation in what follows is either according to the context the splitting of a mother artery into two or more daughter arteries (meaning largo sensu) or the splitting of a mother artery into exactly two daughter arteries (meaning stricto sensu). A bifurcation is represented on FIG. 3.

The parameters relative to a bifurcation to which the method provides access can be divided in two main categories: parameters relative to the location of the bifurcation and parameter of characterization of the bifurcation.

This means that the method for determining recovers two different methods that will be described in what follows: a method for locating and a method for characterizing.

It should be noted that, for clarity, only the use of the method for determining for one bifurcation is presented while keeping in mind that the method for determining is preferably applied for each bifurcation that exists in the cerebral vascular tree.

In addition, it is assumed that the bifurcation to which the method for determining is applied is a bifurcation which is located along the Circle of Willis. Indeed, this location is where 85% of the saccular intra-cranial aneurysms occur which results in a more precise prediction of aneurysm.

Description of the Method for Locating One Bifurcation of the Cerebral Vascular Tree

The method for locating enables first to detect the presence of a bifurcation and second the location of this bifurcation.

It is assumed that images of the cerebral vascular tree of a subject are available to the system 10.

These images are images of a brain of a subject is acquired by an imaging technique.

The imaging technique is, for instance, a micro Computed Tomography Micro-CT.

In such case, the obtained images only comprise the vascular tree and not other elements of the brain. For this, specific imaging agent can be used provided a dissection step has extracted the brain from the skull.

However, other imaging techniques may be considered such as magnetic resonance imaging (MRI).

In such case, both the vascular tree and other elements of the brain are imaged.

In any case, the images form a set of three dimensional (3D) images of the cerebral vascular tree.

The method for locating first comprises a step of processing the 3D images to obtain a three-dimensional skeleton of the tree by using a first technique detailed below.

The processing step includes an operation of binarization of the images.

In such operation, a threshold is used to obtain voxels with an upper value (indicating the presence of blood) and a lower value (no element detected).

The processing step includes an operation of skeletonization.

This operation consists in using an octree structure (a set of 3*3*3 pixels).

When the octree structure is full of voxels with an upper value, the voxels with an upper value linked to the octree structure are set as candidate for elimination.

The candidate voxels are then eliminated after testing that their removal does not affect the connectivity of the skeleton. In case, their removal does not keep the connectivity of the skeleton, the candidate voxels are not removed.

By iteratively repeating this operation and changing the position of the octree structure so as to sweep over all the image, a skeleton is obtained.

At the end of the step of processing, a three-dimensional skeleton of the tree is obtained.

The method also comprises a step of analyzing which is applied on the skeleton by using a second technique.

The second technique enables to obtain a connected and non-oriented graph of the vascular tree.

By definition, a graph is a set of nodes linked by edges.

The second technique consists in detecting the edges of the skeleton and setting that the extremities of each edge is a node.

The method also comprises a step of detecting.

At the step of detecting, bifurcations are detected.

For this, it is detected the configurations of the graph wherein a node is linked to at least three edges (in particular, three edges for a bifurcation and four edges for a trifurcation).

In this method, 3D skeletonization combined with a graph-based approach therefore enables to detect the bifurcations located on the Circle of Willis for a subject.

It can be emphasized that the use of a graph approach allows scanning of the skeleton (and hence the volume) in 3D thus avoiding the two-dimensional (2D) slice-by-slice analysis. This preserves the 3D information that may be lost when projecting from 3D to 2D. The graph also provides an accurate localization of the bifurcation center in the 3D space. Furthermore, the graph can be restricted to locate bifurcations in any region of interest.

As shown after, the validity of the method has already been demonstrated for both mice and human cerebral vasculatures.

In addition, the method for determining enables to locate in an accurate way a bifurcation.

The method can be applied to any size of vessels with similar performance.

It can also be noted that exploiting the full 3D skeleton instead of two-dimensional images slices reduces the total processing time.

Description of the Method for Characterizing the Bifurcation

Once properly located, various geometrical properties of each cerebral artery bifurcation are determined in a step of determining at least a characteristic of the bifurcation.

### Choice of the Parameters by the Applicants

The Applicants have carried out a selection among the parameters characterizing a vascular tree that can infer a risk aneurysm formation.

In the experimental sections, the Applicants have identified six parameters that can be interesting which are the values of bifurcation angles, the sum of bifurcation angles, the cross-section of the mother artery, the difference between daughter arteries cross sections, the geodesic length of the mother artery and the tortuosity of the mother artery.

These parameters can be generalized to bifurcation angles, bifurcation dimensions (including cross-area and thickness), the length of the artery between two consecutive bifurcations and the tortuosity of the artery between two consecutive bifurcations.

Before detailing specific ways of obtaining such parameters as examples, it should be noted that a smart selection was made by the Applicants among possible parameters. For instance, it may have been considered that the total number of bifurcations over the length of the artery be a relevant parameters. Prima facie, the change of diameters of the mother artery may also be a promising parameter in this context.

Some brief phenomenological considerations are developed in what follows so as to show how the Applicants construe the way the parameters may have an influence.

The Applicants imagine that the selected parameters have a relatively important influence on the dynamics of the fluid at the bifurcation.

For instance, a large geodesic gap between bifurcations may result in an increased speed of the blood flow into the mother branch of a bifurcation, hence weakening and distorting the junction vessel wall and leading to the formation of a bulge. As a consequence, a large geodesic distance between two adjacent bifurcations, coupled with a significant mother branch cross-section area and large daughter angles enhances the risk of saccular aneurysm forming.

It should also be mentioned that the experimental section shows that among the geometrical parameters, some of them are of specific relevance.

For the reasons above, at the step of determining characteristics of the bifurcation, it is proposed to obtain the bifurcation angles, their area cross sections, the length of the arteries between consecutive bifurcations, as well as a measure of the arterial tortuosity.

### Bifurcation Angles

A bifurcation angle can be defined as the combination of two angles which will be named [symbol] and [symbol] in FIG. 3.

The first angle [symbol] is the angle between the mother artery and the first artery while the second angle [symbol] is the angle between the mother artery and the second artery.

For obtaining the first angle [symbol] , the system 10 calculates the following formula:

[symbol]

=a tan 2(∥−\vec{CN}⊗\vec{CL}∥₂−\vec{CN}·\vec{CL})

Where:
C is a graph node representing the bifurcation center,
L, M, N are the remaining graph nodes delimiting the branches of the bifurcation,
\vec{CN} is the vector linking C to N,
\vec{CL} is the vector linking C to L,
a tan 2(y,x) is a function that computes arc tangent of all four quadrants providing a result in the interval [−π, π],
⊗ is the symbol of the cross product,
· is the symbol of dot product, and
∥ ∥₂ designates the second norm.

The same method is applied for the estimation of the second angle Â₂.

### Geodesic Distance Between Two Consecutive Bifurcations

By definition, the geodesic distance between two consecutive bifurcations is the geodesic distance of the centers of the two bifurcations.

The geodesic distance between two points of a bifurcation is the length of the path followed in the graph to join both, that is the number of voxels between these two points.

The estimation of the geodesic distance is a precise estimation thanks to the precision of the detecting method detailed previously.

### Arterial Cross-Section

Each bifurcation B of a center C is extracted from the volume within a 60×60×60 block of voxels.

The cross-sectional area is computed by considering a voxel belonging to a mother artery located at a distance of 10 voxels from the bifurcation center C.

For any considered bifurcation, and for each of the arteries composing the bifurcation, the artery's skeleton is aligned along one axis (either x, y or z-axis) of the 3D Cartesian domain. This means that a 3D rotation is performed so as to line up the axis of the artery perpendicularly to a geometrical 2D plane (xy, yz, or xz).

Then, a 2D slice along the chosen plane provides the best representation of the artery cross-section.

Without such an alignment process, the extracted 2D cross-section could represent a slanted section of the artery, thus exaggerating one of its diameters.

The 2D slice of the 3D volume along the determined section is extracted.

Then a contour detection is carried out for eliminating voxels that do not belong to the 2D slice.

The cross-sectional area is the number of pixels which are inside the detected contours.

### Arterial Thickness

Arteries are made of various cellular layers. The innermost layer is called the tunica intima. The innermost layer is in direct contact with the blood flow.

The Applicants choose to define the arterial thickness as the artery thickness within the inner artery wall.

Once the artery has been aligned along the z-axis, and a 2D slice of the TOF volume has been extracted at the step of measuring the arterial cross-section, the method next computes a set of oriented projections of this 2D slice image onto 1D projection bins. A discrete Radon transform is applied. The projections span a 180° range of view angles with a 1° step.

For each of these 180 angles, the width of the projected vessel is computed.

This permits an angle-sensitive determination of both the minimum and maximum thickness of the blood vessel.

After the artery alignment previously explained, the 2D slice is binarized.

The extracted 2D plane consists of a binary oval-like shape of the artery surrounded by a black uniform background (pixels set to zero).

For every angle, the discrete Radon projection sums up the pixels of the 2D plane onto a 1D projection. Each line of pixels being crossed by one orientation “beam” sums up into a projection bin.

Hence, the width of the projection (non-zero bins) represents the width of the artery for this given viewing angle.

If the artery has an imperfect oval shape following a certain inclination, all 180 projections are scanned. Only the two projections presenting the minimum and maximum widths are retained.

### Artery Tortuosity

Given an artery A and its 3D skeleton S_{A}, 3D normal vectors of the skeleton binary voxels are first calculated. Tangent vectors for each voxel in the artery are computed and used to derive the normal vectors. These are oriented perpendicular to the 3D tangent vectors.

To compute the curvature of a target voxel vᵢ, the method comprises a step of assessing variations between its normal vector \vec{n}ᵥ_{ᵢ} and the normal vectors of its neighbors. This assessment consists in considering four voxels at each side (left and right) of the target voxel.

The method also comprises calculating the mean of these normal vectors and their vector positions on each side.

In this calculating operation, the mean is an arithmetical mean.

This enables to obtain two normal vectors and two vector positions at the left and right of the target voxel.

This enables to obtain the curvature C(vᵢ) of a target voxel vᵢ by carrying out a calculation which mathematically expresses as:

$$C ( {v}_{i} ) = \frac{{C}_{left} + {C}_{righ t}}{2}$$

where C_{left} is given by:

$${C}_{left} = \frac{( {\vec{n}}_{{v}_{i}} - mean ( {\vec{n}}_{{v}_{left}} ) ) \cdot ( {\vec{v}}_{i} - mean ( {\vec{v}}_{left} ) )}{{‖ {\vec{v}}_{i} - mean ( {\vec{v}}_{left} ) ‖}_{2}^{2}}$$

Where:

$$• mean ( {\vec{n}}_{{v}_{left}} ) \frac{{\sum }_{i = 0}^{\vert {N}_{left} \vert} {\vec{n}}_{i}}{\vert {N}_{left} \vert} ,$$

N_{left} refers to the cardinality of the neighborhood, and

$$• mean ( {\vec{v}}_{left} ) = \frac{{\sum }_{i = 0}^{\vert {N}_{left} \vert} {\vec{v}}_{i}}{\vert {N}_{left} \vert} .$$

These calculations are carried out for each voxel of an artery and aggregated into a global tortuosity T.

The method then comprises a step of deducing the global tortuosity T over the full extent of the artery by using a pooling achieved by using a weighted Minkowski sum.

As an example, the step for deducing is carried out by calculating:

$$T = {( \frac{{\sum }_{i = 0}^{\vert v \vert} {C ( {v}_{i} )}^{p}}{\vert v \vert} )}^{1 / r}$$

where the weight factors p and r have been empirically set to 49.9 and 23.6 respectively, as these values provided the most accurate estimation of the overall tortuosity. The accuracy of Tortuosity estimations has been evaluated by computing the correlation and Root Mean Square Error between the models' prediction and measurements from neuro-radiologists.

Applications

Therefore, the method for determining is an accurate method for obtaining parameters relative to a bifurcation.

Thus, it has been identified several parameters that correlate strongly with and may help to predict the risk of aneurysm formation.

As explained previously, the parameters are mainly related to the geometry of the bifurcation, showing that the anatomical disposition of the brain vasculature may influence the chance of aneurysm formation.

These parameters can be combined in order to better estimate the risk of occurrence of an intra-cranial aneurysm on a given bifurcation.

Optionally, at the expense of the rapidity, to these parameters, can also be added parameters relative to genetic predisposition and environmental risk factors, such as high blood pressure or smoking habits.

Many applications of this determining method can be considered. Some of them are developed in this section.

Are notably developed the applications linked to aneurysm. When relevant, the applications are in vitro applications.

### Method for Predicting

For this application, it is proposed a method for predicting that a subject is at risk of developing an aneurysm.

The method for predicting comprises a step for carrying out the steps of the method for determining parameters of at least one bifurcation of a cerebral vascular tree of the subject, to obtain determined parameters.

The method for predicting also comprises a step of predicting that the subject is at risk of developing an aneurysm based on the determined parameters.

### Method for Diagnosing

This application corresponds to a method for diagnosing an aneurysm to a subject.

The method for diagnosing comprises carrying out the steps of the method for determining parameters of at least one bifurcation of a vascular tree of the subject, to obtain determined parameters.

The method for diagnosing also comprises carrying out a step of diagnosing aneurysm based on the determined parameters.

### Method for Treating

This application corresponds to a method for treating an aneurysm.

The method for treating comprises carrying out the steps of the method for determining parameters of at least one bifurcation of a vascular tree of a subject, to obtain determined parameters.

The method for treating also comprises carrying out a step of administrating a medicine treating the aneurysm determined based on the determined parameters.

### Method for Identifying a Target

This application corresponds to a method for identifying a therapeutic target for preventing and/or treating an aneurysm.

The method for identifying comprises carrying out the steps of the method for determining parameters of at least one bifurcation of a vascular tree of a first subject, to obtain first parameters, the first subject being a subject suffering from an aneurysm.

The method for identifying also comprises carrying out the steps of the method for determining parameters of at least one bifurcation of a vascular tree of a second subject, to obtain second parameters, the second subject being a subject not suffering from an aneurysm.

The method for identifying further comprises a step of selecting a therapeutic target based on the comparison of the first determined parameters and the second determined parameters.

### Method for Identifying a Biomarker

In this application, a method for identifying a biomarker is proposed.

The biomarker can be one biomarker among a diagnostic biomarker of aneurysm, a susceptibility biomarker of aneurysm, a prognostic biomarker of aneurysm or a predictive biomarker in response to the treatment of aneurysm.

The method for identifying comprises carrying out the steps of the method for determining parameters of at least one bifurcation of a vascular tree of a first subject, to obtain first determined parameters, the first subject being a subject suffering from an aneurysm.

The method for identifying comprises carrying out the steps of the method for determining at least one bifurcation of a vascular tree of a second subject, to obtain second determined parameters, the second subject being a subject not suffering from an aneurysm.

The method for identifying comprises selecting a biomarker based on the comparison of the first determined parameters and the second determined parameters.

### Method for Screening a Compound

This application corresponds to a method for screening a compound.

The medicine has an effect on a known therapeutical target, for preventing and/or treating an aneurysm.

The method for screening comprises carrying out the steps of the method for determining at least one bifurcation of a vascular tree of a first subject, to obtain first determined parameters, the first subject being a subject suffering from aneurysm and having received the compound.

In this context, the term “receive” encompasses any ways of administration of the medicine.

The method for screening also comprises carrying out the steps of the method for determining at least one bifurcation of a vascular tree of a second subject, to obtain second determined parameters, the second subject being a subject suffering from the aneurysm and not having received the compound.

The method for screening further comprises selecting a compound based on the comparison of the first determined parameters and second determined parameters.

Experimental Results

This work of the Applicants is part of a wide national research project named the ICAN project focusing on the understanding of the multiple factors that may encourage the formation of saccular intra-cranial aneurysms along the Circle of Willis. The ICAN project aims at determining the reasons why an aneurysm would appear for a given patient at a particular bifurcation depending of many different factors (such as patient habits, family history, genetic predisposition, bifurcation geometry). The ICAN Project has several active components; a study of the genetics of aneurysm formation, automatic detection of aneurysms and here the automated measurement of arterial properties, specifically around the bifurcations where the aneurysms mostly occur.

Medical image processing tools are thus required.

During the first part of the project, a study was conducted on mice.

The mice were induced into a basal or hypertensive condition (by administering L-NAME in their water) to promote the formation of saccular intra-cranial aneurysms.

Micro Computed Tomography scanner (Micro-CT) acquisition of these mouse brains was then done post-mortem.

In the second part of this project, the imaging tools developed for aneurysm detection were applied to existing Magnetic Resonance Imaging (MRI) acquisitions from human subjects by Time-Of-Flight (TOF).

Commonly, intra-cranial aneurysms are monitored using Computed Tomography Angiography or Magnetic Resonance Angiography (MRA). Cerebral angiography thus displays the full brain vasculature as a 3D digital volume.

In this study, the Applicants used of classical MRA acquisitions on human patients by TOF (MRA-TOF in what follows).

The Applicants tested his method on mice vasculature acquired using the Micro-CT. Mice went through a barium sulphate injection in the vascular tree, prior to a cerebral Micro-CT acquisition.

The accuracy of various measurements was tested using two distinct modalities with significantly different resolutions.

The resolution of the MRA-TOF volumes used in this work were ranging from (290×520×168) to (696×768×168) voxels, with voxels size of respectively 562.5×562.5×500.03 μm³ and 0.9375×0.9375×2.9351 mm³.

The resolution of images acquired with the Micro-CT were (1008×1141×1008) with a pixel size of 12 μm.

The Applicants had at their disposal the Micro-CT acquisition of 22 mice brains. Once the mice were euthanized and injected with contrast agent (barium sulphate), the Micro-CT renders visible fine details of the vascularization in the brain.

The Applicants have collected 39 MRI acquisitions on humans, among which 26 present an unruptured aneurysm (23 saccular aneurysms located onto a bifurcation and 3 fusiform aneurysms).

The Application have validated their method through a comparison with the ground truth. The Applicants have shown that the method is accurate for both Micro-CT and MRA-TOF images.

For the Micro-CT, the subcutaneous tissue is not imaged as the brain was extracted from the skull before image acquisition while it appears in the image obtained by MRA-TOF images.

In MRA-TOF case, a process to separate the cerebral vasculature from the subcutaneous tissue is used.

As an illustration, it can be considered a method named Brain Extraction Tool which comprises establishing an intensity histogram to determine an initial value for brain and non-brain thresholds. The center-of-gravity of the head image is then determined. A triangular tessellation of a sphere's surface is then initialized in the brain and allowed to slowly deform, one vertex at a time so as to reach the brain's edge along with the determined thresholds. The thresholds are modified until the brain's edge is reached.

The Applicants have tested the method for determining bifurcation which is proposed in this specification on different 3D cerebral vasculatures to ensure that all 3D bifurcations located on the Circle of Willis are detected correctly. The proposed method correctly detected all bifurcations of interest (9 bifurcations located on the Circle of Willis). This method was also able to successfully detect all bifurcations within any different region of the full target volume.

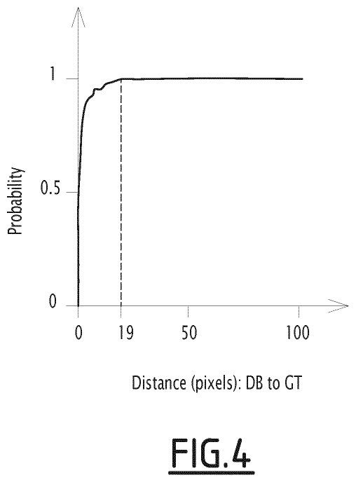

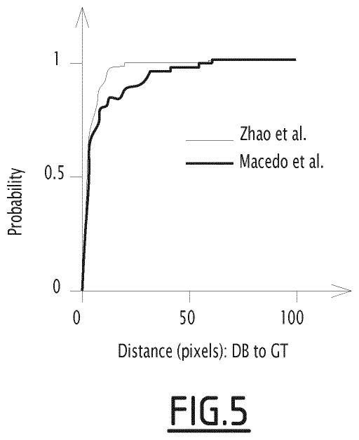

FIGS. 4 and 5 shows cumulative histograms of distances between the predicted bifurcations' localizations named DB and the ground truth localizations named GT for respectively the method detailed in the specification and two methods of the prior art.

The tests were carried out with 10 volumes with 16 bifurcations each belonging to the Vascusynth database. In this database, each group contains 12 randomly generated volumes with the number of bifurcations incrementing from 1 to 56 in steps of 5. 3D coordinates of the bifurcation centers were provided. These locations represent the ground truth coordinates for each bifurcation center.

The two methods of the prior art are the methods of Macedo et al. and Zhao and Hamarneh which are mentioned in the background section.

To compare the three approaches, the Applicants considered a subset of the Vascusynth database consisting in 10 volumes with 16 bifurcations in each volume.

It can be observed that the approach proposed by the Applicants detects all possible bifurcations, with a maximum 19 pixels error from the ground truth centers whereas the two competing approaches achieve bifurcation detection with a 50 pixels shift error. Furthermore, the distribution of distance errors from the proposed approach (FIG. 4) diminishes faster than those for the comparison methods (FIG. 5). This result confirms the high precision of the proposed approach to detect and localize 3D bifurcations.

Concerning now the calculation of bifurcation angles, a comparison was carried out between the proposed method and the value obtained by the software ImageJ which was developed by the NIH in 1987.

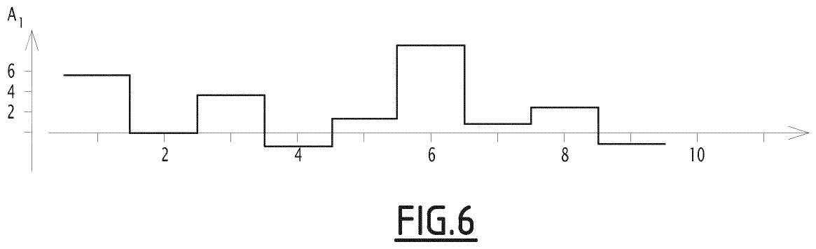

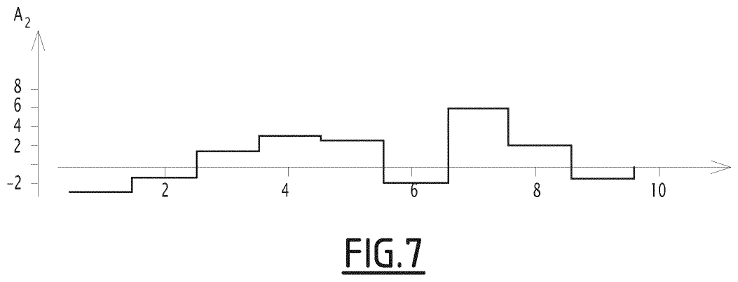

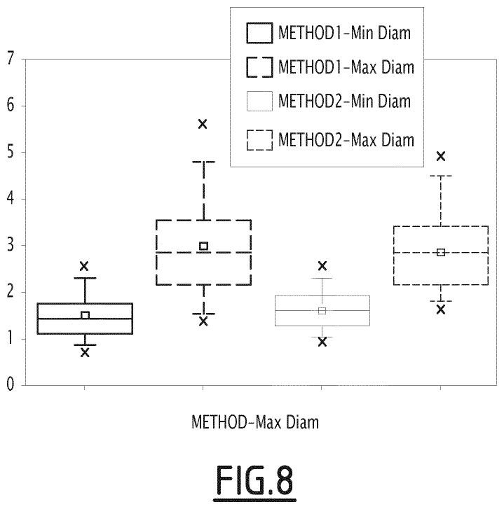

The Applicants manually measured these angles, the Applicants used the average of these two independent observer measures. FIGS. 6 and 7 present the results for the two angles [symbol] and [symbol] and they show the differences between the values obtained by the method and the human measures.

It can be noted that the angle values estimated by the proposed method are highly correlated with the manual measurements.

The differences, being mostly positive, mean that our model slightly overestimates the bifurcation angles. However, the errors are acceptably low over all twenty measurements.

Moreover, using the method, the angles are automatically computed, without requiring any user intervention.

In order to ensure that our model properly estimates arterial diameters, the Applicants have collected subjective thickness measurements. Ten people were asked to manually measure the smaller and larger diameters of ten cerebral arteries. The measurements were performed using the ImageJ software.

The method as described accurately predicted both diameters. The Pearson correlation between the method prediction and the ground truth (user-defined measurements) was 0.93 for the minimum diameters and 0.92 for the maximum diameters.

To the best of the Applicants' knowledges, there are currently no existing methods to measure the global curvature of a cerebral blood vessel. The Applicant is thus unable to compare his tortuosity measurements with observations in the literature.

Thus, the Applicant compared the results of the proposed tortuosity measure against the subjective scores provided by human observers. To verify this, the Applicants have constructed a ground truth which consists of 27 human cerebral arteries. The tortuosity indexes of these branches were evaluated by 20 human observers. The observers were able to interact with the 3D arteries (3D rotation, zoom in/zoom out) and were asked to assign a representative tortuosity score between 0 and 100 (where 0 refers to a low tortuosity degree while 100 represents extreme tortuosity).

The objective tortuosity results provided by the method and the subjective results collected from human observers showed strong agreement, with a Pearson correlation coefficient of 90.03% and a normalized root mean square error between the objective and subjective results of 0.13. These indicators attest the precision of the proposed measure of tortuosity.

All the parameters whose determination has been previously described enable to characterize arterial bifurcation geometry. It is shown in what follows that there is a link between arterial bifurcation geometry and the risk of aneurysms.

The risk of aneurysm formation depends on several factors. A genetic predisposition may account for a significant portion of the probability of aneurysm formation. Environmental interactions, such as smoking habits or hypertension may also increase the risk of developing an aneurysm. The Applicants wished to avoid the complications and interference from these external factors on his study of the influence of the bifurcation geometry on aneurysm formation. To do this, the Applicants analyzed the effects of arterial geometry when those external factors remain constant by analyzing intra-patient examples. The Applicants thus compared the geometry of matching bifurcations located on the same arteries on the left and right side of the patient's brain (mirror bifurcations) to evaluate the risk of aneurysms. The Applicants gathered the MRA-TOF volumes from 10 patients. Among the 10 patients, four presented an aneurysm on the left middle cerebral artery whereas the others exhibited an aneurysm on the right middle cerebral artery. The proposed method for determining parameters of the bifurcation was tested on this data base of 20 bifurcations.

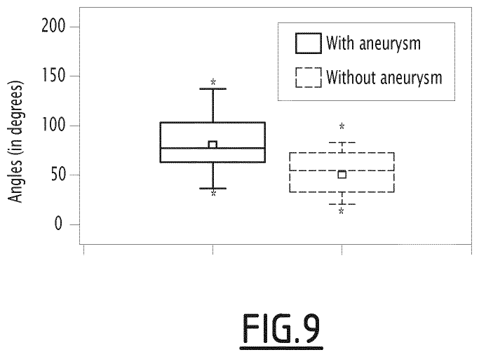

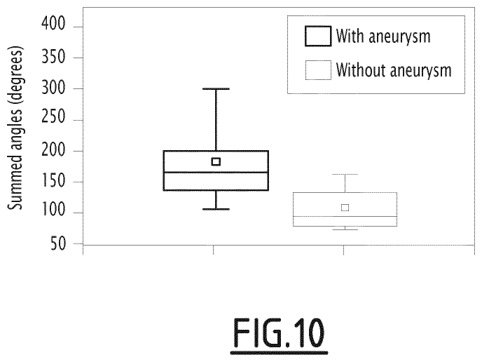

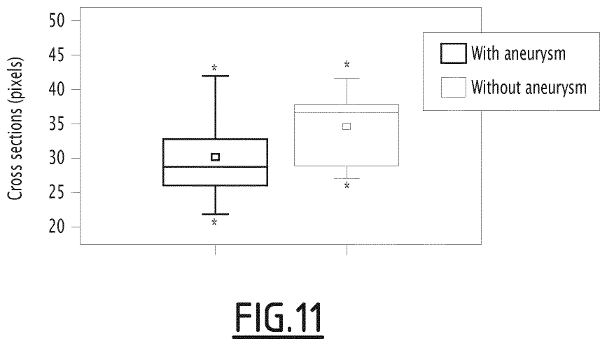

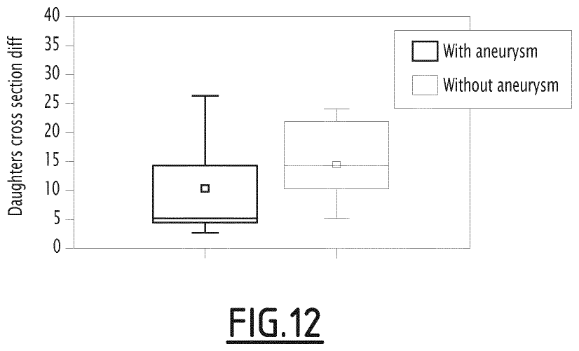

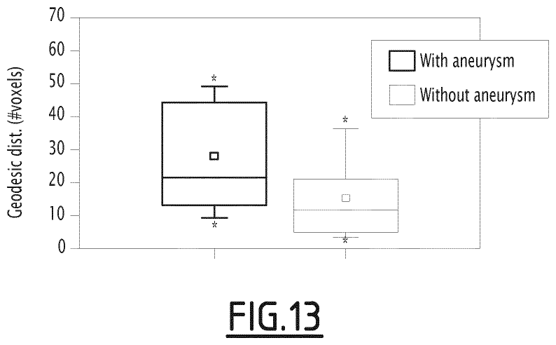

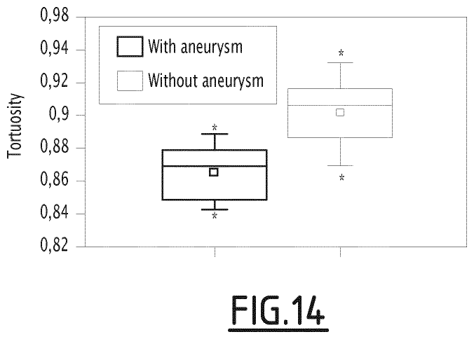

FIGS. 9 to 14 shows the geometrical parameters obtained by carrying out the method with aneurysm (boxes in full line or made of darker lines according to the considered figure) and without aneurysm (dotted boxes or made of lighter lines according to the considered figure): FIG. 9 corresponds to the combination of both bifurcation angles, FIG. 10 to the sum of both bifurcation angles, FIG. 11 to the cross-section of the mother artery, FIG. 12 to the difference between daughter's cross-sections, FIG. 13 to the geodesic length of the mother artery and FIG. 14 to the tortuosity of the mother artery.

As expected, the bifurcations that actually exhibited an aneurysm presented larger angles, lower tortuosity, and longer distance of the mother artery.

However, as can be seen on FIG. 11, it appears that the cross-section of the mother artery may not be a strong feature to help estimate the risk of occurrence of an aneurysm.

For each bifurcation, the Applicants also subsequently computed the difference between the cross-sections of the two daughter arteries. A significant difference between daughters' arterial cross-section did not seem to be related with the occurrence of an aneurysm.

Overall, except for the diameter feature which, on the panel of subjects, could not assert a link with the risk of aneurysm formation, a strong relationship was found for all three other features. Importantly, the results from our proposed graphic-based model closely mimic the chances of occurrence of an aneurysm based on three geometric considerations: the angle formed by the daughter branches, the tortuosity and length of the mother branch.

The measured vessel angles, diameters and tortuosity estimations were well correlated to the measurements made by human observers. Full characterization of these bifurcations enabled us to associate these features with the possible occurrence of a saccular aneurysm. Our results show, given control of other contributing factors (for genetic differences and environmental factors), that bifurcation geometry can be stated to play an important role in the risk of developing an aneurysm. Amongst the geometric features the Applicants quantified, the bifurcation angles, the tortuosity and the distance to the previous bifurcation appeared to be the three most important.

Additional Elements on the Graph-Based Detection

The non oriented graph G=(V,E,w) is constructed on the entire 3D skeleton where:
V represents the set of nodes,
E∈V×V the set of edges and
w(vᵢ,vⱼ) the weight of the edge e(vi,vj)∈E connecting the nodes vᵢ and vⱼ.

Similarly to S, to each node vi∈V of the graph are associated its 3D coordinates vᵢ=(x, y, z).

A weighted graph is a graph wherein the edges have been assigned with a weight. Graphs with weights, or weighted graphs, are used to represent structures in which pairwise connections have some numerical values.

In this context, using a weighted graph rather than a graph is advantageous in so far as this enables to filter each edge with small weight which often corresponds to artefact(s) or to vessel(s) deprived of bifurcations.

Therefore, using a weighted graph is preferable since the detection of bifurcation (and also the determination of properties of the bifurcation) is better.

Such use of a weighted graph is a clear difference with results and methods from other groups.

Notably, document WO 03/034337 A2 describes a method for analysing an object data set in which a tubular structure having a plurality of branches and bifurcations occurs, wherein said object data set assigns data values to positions in a multi-dimensional space, which data values relate to an object to be examined, and wherein the positions along the branches and of the bifurcations of said tubular structure are labelled, each branch and bifurcation having a unique label. In order to improve accuracy when applying the disclosed method particularly for fully-automated vessel tracing, particularly in the vessel structure of the brain of a patient, the following steps are proposed: selecting a starting point in or near a branch or bifurcation of interest of the tubular structure, orienting a probe comprising a sphere and a plane through the centre of the sphere around said starting point such that the plane goes through said starting point, adjusting the orientation of the probe such that the plane is orthogonal to the tubular structure and that the centre of the sphere is on the central axis of the tubular structure, thereby using surface vertices inside the sphere, said surface vertices being labelled according to the label of the nearest position along the branches and of the bifurcations, wherein only surface vertices are used for adjusting the orientation of the probe having a label equal to the label of the branch or bifurcation of interest or of the next bifurcation or extremity along the branch of interest.

This method disclosed in document WO 03/034337 A2 relies on the use of root points and seed points to determine a circulation sense in an artery. There is thus no use of a weighted graph which results in a poorer detection.

Similarly, document WO 2015/059706 A2 also deals with a vascular characteristic determination method which does not provide a good detection.

This is notably the case because the use of a weighted graph is not suggested.

Additional Elements on the Branches Area Sections

Yet another significant anatomical property that may explain the occurrence of intra-cranial aneurysms is the thickness of the arteries. Considering a bifurcation B of center v_{c} in the 3D skeleton S, the inventors have delimited the latter with three voxels A₁, A₂ and A₃ located at a distance of 30 voxels from the bifurcation center v_{c} on each branch. The aim is to compute three discrete plans P₁, P₂ and P₃ in the 3D space which will cut each respective branch (b1, b2, b3) of the bifurcation B through three respective skeleton voxels A₁, A₂ and A₃.

Suppose that one has to compute the discrete plan P₁ cutting the branch b1 through the voxel A₁. For this, a voxel A_{1N}∈b1 nearby A₁ is selected (a distance equal to seven voxels is chosen but a length between 3 and 10 voxels can be chosen) in order to have a cut plane P₁ tangent to the voxel A₁ and perpendicular to the vector A₁A_{1N}. Once the voxels A₁ and A_{1N} determined, the cut plan can be computed according to the following algorithm:

AB ← (Bₓ−Aₓ, B_{y}−A_{y}, B_{z}−A_{z})
X ← [1 : 120]
Y ← [1 : 120]
Indicesplan ← List (Indices of voxels constituting the cut plan)
for each i in X do

for each j in Y do

z ← (−(ABₓ*(i−Aₓ)) − (−(AB_{y}*(j−A_{y}))/ ABₓ + Aₓ
if z < 120 then

z= └z┘
indicesplan.push(l,j,z)

end if

end for

end for
return indicesplan

Such routine enables to obtain a cut plane.

The voxels located at the intersection between the 3D skeleton and the discrete plane represent the cross section of the branch. These can be easily selected since the 3D skeleton as well as the plan are represented as 3D matrices. The area section is then simply obtained by counting the intersection vertices.

In order to cope with branches having different localized cross sections areas, three different cut planes can be computed with three different cross section areas. The final branch cross section area is defined as the average of the three computed areas. The average is, for instance, an arithmetic average. The number of cross section areas may also be higher, notably superior or equal to 5.

In some cases, the angle between the daughter branches of a 3D bifurcation is relatively small. This leads to a plane cutting the bifurcation through two branches. The aim now is to distinguish between the intersection voxels related to each branch in order to compute their different cross section areas. To tackle the problem, a simple and fast clustering approach is used. Having the coordinates of the voxel A through which the plane cuts the bifurcation, the Euclidean distance Dᵢ between A and all the intersection voxels is computed and outputted. Afterwards, a clustering method is applied on the inter-voxels distances to separate the two branches.

1. Method for determining at least one parameter of at least one bifurcation of a vascular tree of a subject, notably a cerebral one, the method being computer-implemented, the method comprising the steps of:
processing a three-dimensional image of the vascular tree with a first technique to obtain a three-dimensional skeleton of the tree,
analyzing the obtained skeleton by a second technique to obtain a graph of the vascular tree, the graph being a set of nodes linked by edges, and
detecting the presence of a bifurcation when the graph comprises a node linked to at least three edges.

2. Method according to claim 1, wherein, at the step of analyzing, the graph is a set of nodes linked by edges with a weight.

3. Method according to claim 1, wherein the method further comprises a step of calculating the geodesic distance between two bifurcations.

4. Method according to claim 1, wherein the method for determining further comprises a step of determining a characteristic of the bifurcation.

5. Method according to claim 4, wherein a bifurcation angle is defined for a bifurcation and a cross-section area is defined for a bifurcation, the step of determining comprising determining the bifurcation angle of the detected bifurcation and/or the cross-section area of the detected bifurcation.

6. Method according to claim 3, wherein the method comprises a step of obtaining the artery tortuosity, the step of obtaining comprising calculating the curvature of each voxel of an artery.

7. Method according to claim 6, wherein the step of obtaining comprises pooling the curvatures to obtain the tortuosity parameter.

8. Method according to claim 7, wherein the pooling is achieved by using a weighted Minkowski sum.

9. A method for predicting that a subject is at risk of developing an aneurysm, the method for predicting at least comprising the steps of:
carrying out the steps of a method for determining at least one parameter of at least one bifurcation of the vascular tree of the subject, to obtain determined parameters, the method for determining being according to claim 1, and
predicting that the subject is at risk of developing the aneurysm based on the determined parameters.

10. A method for diagnosing an aneurysm, the method for diagnosing at least comprising the steps of:
carrying out the steps of a method for determining at least one parameter of at least one bifurcation of the vascular tree of the subject, the method for determining being according to claim 1, and
diagnosing the aneurysm based on the determined parameters.

11. A method for identifying a therapeutic target for preventing and/or treating an aneurysm, the method comprising at least the steps of:
carrying out the steps of a method for determining at least one parameter of at least one bifurcation of the vascular tree of a first subject, to obtain first determined parameters, the method for determining being according to claim 1 and the first subject being a subject suffering from the aneurysm,
carrying out the steps of the method for determining at least one parameter of at least one bifurcation of the vascular tree of a second subject, to obtain second determined parameters, the method for determining being according to claim 1 and the second subject being a subject not suffering from the aneurysm, and
selecting a therapeutic target based on the comparison of the first and second determined parameters.

12. A method for identifying a biomarker, the biomarker being a diagnostic biomarker of an aneurysm, a susceptibility biomarker of an aneurysm, a prognostic biomarker of an aneurysm or a predictive biomarker in response to the treatment of an aneurysm, the method comprising at least the steps of:
carrying out the steps of a method for determining at least one parameter of at least one bifurcation of the vascular tree of a first subject, to obtain first determined parameters, the method for determining being according to claim 1 and the first subject being a subject suffering from the aneurysm,
carrying out the steps of the method for determining at least one parameter of at least one bifurcation of the vascular tree of a second subject, to obtain second determined parameters, the method for determining being according to claim 1 and the second subject being a subject not suffering from the aneurysm, and
selecting a biomarker based on the comparison of the first and second determined parameters.

13. A method for screening a compound useful as a probiotic, a prebiotic or a medicine, the compound having an effect on a known therapeutical target, for preventing and/or treating an aneurysm, the method comprising at least the steps of:
carrying out the steps of a method for determining at least one parameter of at least one bifurcation of the vascular tree of a first subject, to obtain first determined parameters, the method for determining being according to claim 1 and the first subject being a subject suffering from the aneurysm and having received the compound,
carrying out the steps of the method for determining at least one parameter of at least one bifurcation of the vascular tree of a second subject, to obtain second determined parameters, the method for determining being according to claim 1 and the second subject being a subject suffering from the aneurysm and not having received the compound, and
selecting a compound based on the comparison of the first and second determined parameters.

14. A computer program product comprising instructions for carrying out the steps of a method according to claim 1 when said computer program product is executed on a suitable computer device.

15. A computer readable medium having encoded thereon a computer program according to claim 14.
